(19) United States
(12) Patent Application Publication
(10) Pub. No.: US 2024/0075001 A1
(43) Pub. Date: Mar. 7, 2024

(54) METHODS TO REDUCE HEMORRHAGE AND MORTALITY ON THE BATTLEFIELD AND APPLICATIONS FOR TRAUMA PATIENTS

(71) Applicant: Darren Rubin, Largo, FL (US)

(72) Inventor: Darren Rubin, Largo, FL (US)

(21) Appl. No.: 18/187,906

(22) Filed: Mar. 22, 2023

Related U.S. Application Data
(60) Provisional application No. 63/345,199, filed on May 24, 2022.
(60) Provisional application No. 63/393,645, filed on Jul. 29, 2022.
(60) Provisional application No. 63/375,258, filed on Sep. 12, 2022.

(51) Int. Cl. A61K 31/195; A61K 9/00; A61K 45/06

(52) U.S. Cl. CPC A61K 31/195; A61K 9/0019; A61K 45/06

(57) ABSTRACT

Methods and applications of the present disclosure include promoting blood clotting, controlling hemorrhage, and or inhibiting fibrinolysis in soldiers and civilians having experienced massive bleeding from a wound by administering antifibrinolytic agents, such as tranexamic acid, and or one or more procoagulants, in preferably autoinjector format.

### CROSS-REFERENCE TO RELATED APPLICATIONS

This application claims priority to U.S. Provisional Application Ser. No. 63/345,199 filed May 24, 2022, entitled “Methods To Reduce Hemorrhage and Mortality On The Battlefield And Applications For Trauma Patients,” prior U.S. Provisional Application Ser. No. 63/393,645 filed Jul. 29, 2022, entitled “Methods To Reduce Hemorrhage and Mortality On The Battlefield And Applications For Trauma Patients,” and prior U.S. Provisional Application Ser. No. 63/375,258 filed Sep. 12, 2022, entitled “Methods To Reduce Hemorrhage and Mortality On The Battlefield And Applications For Trauma Patients,” the entire disclosure of which is hereby incorporated by reference in its entirety into the present disclosure.

### SPECIFICATION BACKGROUND

Soldiers wounded on the battlefield or by an improvised explosive device (IED) can experience massive blood loss that can result in hemorrhagic shock or death. There is a great need for ways to reduce soldier mortality from hemorrhage and to enhance survivability following blood transfusion. There is also a great need to help civilian patients wounded in a vehicle accident or other trauma associated with hemorrhage. There is also a lack of hemostatic agents that can be self-administered or administered by those not medically trained, e.g., soldiers, police, firemen, until medical help can arrive. Without hemostatic agents that can be administered more quickly than conventional methods (methods that require an intravenous line), before or while an intravenous infusion is being established, wounded soldiers and civilians can perish. A solution is needed to keep the soldier or patient alive long enough to survive transport to be treated at a hospital or medical facility.

### DETAILED DESCRIPTION

This disclosure includes methods and applications to promote blood clotting, control hemorrhage, and or inhibit fibrinolysis in soldiers and civilians having experienced massive bleeding from a wound by administering antifibrinolytic agents, e.g., tranexamic acid and or aminocaproic acid, and or one or more procoagulants, e.g., zeolites, and or other hemostatic agents, in one or more pharmaceutically acceptable formulations, prior to or simultaneous with blood volume replacement or blood transfusion. Such methods may reduce the need or amount of blood transfusion. Wounds often associated with massive bleeding include gunshot and knife wounds, as well as, wounds caused by IEDs and vehicle crashes. Such methods can help stabilize a hemorrhaging patient so that he or she can survive transport and or survive long enough to be treated by medical staff. Some methods provide the ability for self-administration or administration by nonmedical staff, for instance, when medical staff or other medical supplies are not available out in the field or battlefield.

A bullet wound is an example of a hemorrhagic wound. The firing of a bullet at a target causes ballistic trauma, otherwise known as a gunshot wound or bullet wound. A penetrating bullet causes a disruption in tissue and a cavitation in the body, which is associated with severe bleeding or hemorrhage. Significant loss of blood often causes hypovolemic shock marked by diminished blood pressure, decreased organ perfusion and inadequate delivery of oxygen. If bleeding cannot be stopped, bleeding to death or bleeding out, otherwise known as exsanguination, can occur with the loss of even half the body's blood volume.

The basic method of treating a bullet wound until help can arrive is to cover the wound opening with a cloth and apply pressure to the wound to help stop the bleeding and form a clot. Yet, this alone may not be effective. With a bullet wound, it is very possible for a target to bleed out before emergency care can arrive and transfuse blood products and surgically repair the wound; even if emergency care can arrive or have access to the patient in a timely manner, which is not always the case. While applying external compression with bandages to a projectile wound has been used for many centuries, the modern era has seen the elucidation of the blood coagulation or clotting cascade and the discovery of hemostatic agents; agents that control and arrest bleeding to achieve hemostasis, such as by promoting the formation of blood clots. A substance that is hemostatic serves to reduce or stop bleeding.

A number of hemostatic agents exist, including: natural and synthetic clay and silicate materials such as zeolite, kaolinite, and diatomaceous earth; glass and glass-ceramics; polymeric polysaccharides such as algae and shellfish derived chitin, chitosan, and alginate; polymeric proteins; other polymers, such as polyacrylates; glass powders, beads or fibers, such as surface reactive glass-ceramics; and plasma-derived or recombinant clotting factors, such as thrombin, fibrinogen and fibrin; even synthetic nanoparticles and nanofibers. These hemostatic agents may be in the form of powders, gels, or impregnated into gauze bandages and other dressings.

Desmopressin may help coagulation by activating arginine vasopressin receptor 1A, and vitamin K is a factor that helps coagulation. Some embodiments include one or more of these agents.

Antifibrinolytic agents, such as aminocaproic acid and tranexamic acid, prevent blood clots from degrading. Antifibrinolytic agents are analogs to the amino acid lysine and reversibly bind to sites on the proenzyme plasminogen to decrease its conversion to plasmin, which would otherwise degrade fibrin, a fibrous protein involved in the blood clotting matrix.

The present disclosure provides methods of applying hemostatic agents, procoagulant agents and or antifibrinolytic agents, to a wounded soldier, or bleeding civilian, into the bloodstream and or into the tissue near the wound or in the appendage having the wound.

The preferred embodiment of this invention includes one or more autoinjectors containing one or more antifibrinolytic agents, e.g., tranexamic acid and or aminocaproic acid, and or one or more procoagulants, e.g., zeolites, and or other hemostatic agents, in one or more pharmaceutically acceptable formulations. In this embodiment, the hemostatic agents can be self-administered or administered by those not medically trained, e.g., soldiers, police, firemen, until medical help can arrive. This may enable the hemostatic agents to be administered more quickly than conventional methods (methods that require an intravenous line), before or while an intravenous infusion is being established. Such methods may keep the soldier or patient alive long enough to survive transport to be treated at a hospital or medical facility. The preferred embodiment of these autoinjectors include liquid formulations. In some embodiments the autoinjector is powered by a spring mechanism. In other embodiments, the autoinjector is powered by compressed gas. In other embodiments, the liquid is contained in a cartridge, while in other embodiments the liquid is contained in a prefilled syringe. In other embodiments a prefilled syringe is administered manually. In yet still other embodiments, the prefilled syringe is administered with the aid of a syringe assist device that the prefilled syringe fits into, also known as a manual assist device. In some embodiments the injector contains a needle. In other embodiments the injector is needle-free and penetrates the skin with compressed gas or air.

Other embodiments of an autoinjector include one or more nonliquid or gel or powder-like formulations of one or more antifibrinolytic agents and or one or more hemostatic/procoagulant agents that are administered below the dermal layer and begin to mix or dissolve in bodily fluids like blood.

In yet still other embodiments, the one or more antifibrinolytic agents and or one or more hemostatic/procoagulant agents are contained in a solid form, such as a capsule or tablet, or even a drug eluting stent or rod, that gets implanted into the tissue or blood vessel inside or near the wound or appendage. The solid form formulation(s) can be implanted manually by hand or by the use of an injector or autoinjector. The solid form will mix with and or dissolve in bodily fluid such as blood to impart its pharmacologic effect. In some embodiments, the one or more solid form formulation(s) is an implant.

Other methods of administration include administration by intramuscular or intravenous bolus injection, or intravenous infusion, and one or more formulations are contained in a prefilled syringe, vial, cartridge, bottle, or intravenous fluid bag. Other disposable containers can be envisioned for these purposes, and these examples are not meant to be limited. Some embodiments include administration with a portable infusion pump for prolonged intramuscular and or intravenous administration.

Other methods of administration include one or more intraosseous formulations and or one or more intraosseous delivery devices.

Other formats and or combinations of formulations and or other formats of delivery devices and or other routes of delivery of antifibrinolytic agents and or hemostatic/procoagulant agents are possible and the present disclosure is not intended to be limited to the embodiments shown herein, but is to be accorded the widest scope possible consistent with the principles and novel features as previously described.

An example of a first embodiment is an autoinjector containing tranexamic acid. The simplest formulation is an aqueous one. Each autoinjector would contain about 0.5 grams of tranexamic acid in about 3 mL of water. The only excipient is water in the simplest example. In another nonlimiting example, each autoinjector would contain about 0.5 grams of tranexamic acid in about 2.25 mL of formulation volume with water and or one or more additional excipient ingredients. In another nonlimiting example, each autoinjector would contain about 1 gram of tranexamic acid in about 4.5 mL of formulation volume with water and or one or more additional excipient ingredients.

Treatment with one, two, or multiple, tranexamic acid autoinjectors should begin within several hours after sustaining a hemorrhagic injury, and preferably, within 4 hours after sustaining a hemorrhagic injury, and more preferably, within 3 hours after sustaining a hemorrhagic injury, and more preferably, within 2 hours after sustaining a hemorrhagic injury, and more preferably, within 1 hour after sustaining a hemorrhagic injury, and most preferably as soon as is possible or immediately after sustaining a hemorrhagic injury or suspected hemorrhagic injury. One or more grams of tranexamic acid should be provided to the soldier or patient in this manner. Alternatively, one or more grams of tranexamic acid should be provided to the soldier or patient in this manner prior to, during, and or following blood transfusion(s).

An example of a first method for administering the first embodiment tranexamic acid autoinjector is to administer two autoinjectors containing at least 0.5 grams of tranexamic acid each either immediately sequentially, or within 5 to 10 minutes of each other, or both within 1 hour, and preferably both within 1 hour after sustaining the hemorrhagic injury. Optionally, an additional two tranexamic acid autoinjectors are administered within 8 hours after sustaining the hemorrhagic injury or after treatment begins.

An alternative method for administering the tranexamic acid autoinjectors is to administer four autoinjectors containing 0.5 grams of tranexamic acid each within 8 hours after sustaining the hemorrhagic injury, and preferably administering the first two tranexamic acid autoinjectors within the first hour after sustaining the hemorrhagic injury. The second two tranexamic acid autoinjectors can be administered for example at 3 hours and 6 hours after sustaining the hemorrhagic injury, or at 4 hours and 8 hours after sustaining the hemorrhagic injury, for example. The second two tranexamic acid autoinjectors can be administered at least 1 hour apart of each other, or at least 2 hours apart of each other, or at least 3 hours apart of each other, or at least 4 hours apart of each other. The first of the second two tranexamic acid autoinjectors can be administered at least 1 hour apart of the last of the first two tranexamic acid autoinjectors, or at least 2 hours apart of the last of the first two tranexamic acid autoinjectors, or at least 3 hours apart of the last of the first two tranexamic acid autoinjectors, or at least 4 hours apart of each other. The second two tranexamic acid autoinjectors can alternatively be administered immediately sequentially somewhere between 2 hours and 8 hours after sustaining the hemorrhagic injury or after treatment begins. These examples are not meant to be limiting and the inventive methods can be carried out in other ways.

In other embodiments, the at least one tranexamic acid autoinjector contains more than 0.5 grams of tranexamic acid.

In preferred embodiments, the first two tranexamic acid autoinjectors are administered before the patient receives a blood transfusion and or fluid volume replacement therapy.

In other embodiments, the first two tranexamic acid autoinjectors are administered while the patient receives a blood transfusion and or fluid volume replacement therapy.

Other embodiments can include different concentrations, and or different volumes, and or additional excipients in the tranexamic acid autoinjector. For example, a second embodiment can include three autoinjectors containing tranexamic acid, each autoinjector containing about 333.33 mg of tranexamic acid in about 2 mL of water, including about 14.6 mg sodium chloride in about 2 mL, and about 0.914 mg sodium metabisulfite in about 2 mL, and some hydrochloric acid or sodium hydroxide. Analogous methods of administration would pertain to these three autoinjectors, so that the first three autoinjectors are administered preferably within 1 hour after sustaining the hemorrhagic injury, and optionally, three additional autoinjectors are administered preferably within 8 hours after sustaining the hemorrhagic injury or after treatment begins.

Other embodiments can include different concentrations, and or different volumes, and or additional excipients in the tranexamic acid autoinjector. For example, a third embodiment can include four autoinjectors containing tranexamic acid, each autoinjector containing about 250 mg of tranexamic acid in about 1.5 mL of water, including about 11 mg sodium chloride in about 1.5 mL, and about 0.69 mg sodium metabisulfite in about 1.5 mL, and some hydrochloric acid or sodium hydroxide. Analogous methods of administration would pertain to these four autoinjectors, so that the first four autoinjectors are administered preferably within 1 hour after sustaining the hemorrhagic injury, and optionally, four additional autoinjectors are administered preferably within 8 hours after sustaining the hemorrhagic injury or after treatment begins.

In yet other embodiments, the tranexamic acid is in powder form, or a concentrated mixture form, and requires mixing with water, sterile water, water for injection, and or saline prior to use. In some embodiments, a dual chamber cartridge, vial, or syringe contains both tranexamic acid powder (or a concentrated mixture) and aqueous fluid separated until immediate use; whereby the breaking or opening of the separating material allows the tranexamic acid to mix and dissolve in the aqueous fluid, although some agitation may be required.

In other embodiments the tranexamic acid formulation also contains a blood clot-promoting, hemostatic agent; a procoagulant. The at least one procoagulant agent is selected from natural and synthetic clay and silicate materials such as zeolite, kaolinite, and diatomaceous earth; glass and glass-ceramics; polymeric polysaccharides such as algae and shellfish derived chitin, chitosan, and alginate; polymeric proteins; other polymers, such as polyacrylates; glass powders, beads or fibers, such as surface reactive glass-ceramics; and plasma-derived or recombinant clotting factors, such as thrombin, fibrinogen and fibrin; even synthetic nanoparticles and nanofibers.

Other pharmaceutically acceptable excipients or solvents may be included in the formulation, such as acids or bases to adjust pH, or salts to adjust osmolarity, preservatives, stabilizers, or even other solvents, carriers, and polymers, as well as, suspending agents, dispersants, surfactants, crystallization inhibitors, freeze inhibitors, viscosity adjusting agents, or a combination thereof. Crystallization inhibitors are excipients that reduce or prevent the crystallization and or precipitation of solids in the formulation. Freeze inhibitors are excipients that lower the freezing point of the formulation to reduce or prevent freezing, which is important when injecting the formulation in artic field conditions or during a cold winter. Example excipient ingredients may include glycerin, polyethylene glycol(s), propylene glycol, other glycols; polysorbates; cyclodextrins; one or more polymers, such as carbomers, microcrystalline cellulose, methylcellulose, hydroxypropyl-methylcellulose, carboxymethylcellulose and its salts; other salts like sodium chloride and potassium bicarbonate; detergents and/or chelators such as EDTA; certain polymers; pH adjustors (e.g., acids or bases); micelles and or liposomes, preservatives, solvents or water. Pharmaceutically acceptable excipients are preferred. These classes of excipients and/or excipient examples are not meant to be limiting to one skilled in the art. One or more active ingredients, excipients, formulations, or a combination thereof may be in micronized form or nanoparticle form. For example, the formulation of tranexamic acid may contain microspheres and or be in a micro-emulsion or nano-emulsion, or a micro-suspension or nano-suspension, or a micro-colloid or nano-colloid. These examples are not meant to be limiting. Various embodiments may have various viscosities when injecting one or more various formulations.

In another embodiment, tranexamic acid is delivered by one or more autoinjectors to reduce or prevent hemorrhage in hemophiliacs, including those prior to having a dental or medical procedure, and or including those hemophiliacs following a dental or medical procedure. In alternative embodiments, multiple tranexamic autoinjectors are administered to a hemophiliac over the course of two or more days.

In some embodiments, tranexamic acid is delivered by a reusable pen or reusable autoinjector device associated with replaceable containers containing tranexamic acid.

### EXAMPLES

Example 1 is a method, comprising: administering at least two autoinjectors containing tranexamic acid, to a soldier or civilian having a hemorrhagic wound.

Example 2, the subject matter of Example 1 additionally or alternatively includes, wherein at least one of the at least two autoinjectors contains tranexamic acid in an aqueous solution.

Example 3, the subject matter of any of Examples 1-2 additionally or alternatively includes, wherein at least one of the at least two autoinjectors contains tranexamic acid inside an acceptable pharmaceutical formulation containing at least one excipient ingredient in addition to water.

Example 4, the subject matter of any of Examples 1-3 additionally or alternatively includes, wherein at least one of the at least two autoinjectors contains an at least one procoagulant agent or hemostatic agent in addition to tranexamic acid.

Example 5, the subject matter of any of Examples 1-4 additionally or alternatively includes, wherein at least one of the at least two autoinjectors contains an at least one active pharmaceutical ingredient in addition to tranexamic acid.

Example 6, the subject matter of any of Examples 1-5 additionally or alternatively includes, wherein at least one of the at least two autoinjectors contains up to 500 mg or more of tranexamic acid.

Example 7, the subject matter of any of Examples 1-6 additionally or alternatively includes, wherein at least two of the at least two autoinjectors contains equal amounts of tranexamic acid.

Example 8, the subject matter of any of Examples 1-7 additionally or alternatively includes, wherein at least two of the at least two autoinjectors contains different amounts of tranexamic acid.

Example 9, the subject matter of any of Examples 1-8 additionally or alternatively includes, wherein at least two of the at least two autoinjectors contains about 500 mg of tranexamic acid each.

Example 10, the subject matter of any of Examples 1-9 additionally or alternatively includes, wherein at least three of the at least two autoinjectors contains about 333.33 mg of tranexamic acid each.

Example 11, the subject matter of any of Examples 1-10 additionally or alternatively includes, wherein at least four of the at least two autoinjectors contains about 250 mg of tranexamic acid each.

Example 12, the subject matter of any of Examples 1-11 additionally or alternatively includes, wherein at least two of the at least two autoinjectors are administered within about 1 hour after sustaining the hemorrhagic injury and or prior to receiving a blood transfusion.

Example 13, the subject matter of any of Examples 1-12 additionally or alternatively includes, wherein at least four of the at least two autoinjectors are administered within 8 hours after sustaining the hemorrhagic injury or after treatment begins.

Example 14, the subject matter of any of Examples 1-13 additionally or alternatively includes, wherein at least four of the at least two autoinjectors are administered within 8 hours after sustaining the hemorrhagic injury or after treatment begins, and further wherein the first at least two of the at least two autoinjectors are administered within about 1 hour after sustaining the hemorrhagic injury or after treatment begins.

Example 15, the subject matter of any of Examples 1-14 additionally or alternatively includes, wherein at least four of the at least two autoinjectors are administered within 8 hours after sustaining the hemorrhagic injury or after treatment begins, and further wherein the first at least two of the at least two autoinjectors are administered within about 1 hour after sustaining the hemorrhagic injury or after treatment begins, and further wherein the second at least two of the at least two autoinjectors are administered within 7 hours, but not within the first hour, after treatment begins.

Example 16, the subject matter of any of Examples 1-15 additionally or alternatively includes, wherein at least one of the at least two autoinjectors contains an at least one procoagulant agent or hemostatic agent in addition to tranexamic acid; said at least one procoagulant agent is selected from natural and synthetic clay and silicate materials such as zeolite, kaolinite, diatomaceous earth; glass and glass-ceramics; polymeric polysaccharides, chitin, chitosan, alginate; polymeric proteins; polymers, polyacrylates; glass powders, beads, fibers, surface reactive glass-ceramics; and or plasma-derived or recombinant clotting factors, thrombin, fibrinogen fibrin; synthetic nanoparticles, nanofibers, or any combination thereof.

Example 17, the subject matter of any of Examples 1-16 additionally or alternatively includes, wherein said method provides at least one of promoting blood clotting, controlling hemorrhage, reducing blood transfusion, and or inhibiting fibrinolysis in soldiers and civilians having experienced or experiencing hemorrhage from a wound.

Example 18, the subject matter of any of Examples 1-17 additionally or alternatively includes, wherein at least two of the at least two autoinjectors are supplied in a kit containing at least two autoinjectors each containing an injectable formulation of tranexamic acid comprising more than 100 mg/mL of tranexamic acid and up to 167 mg/mL of tranexamic acid.

Example 19 is an injectable formulation of tranexamic acid comprising more than 100 mg/mL of tranexamic acid and up to 167 mg/mL of tranexamic acid.

Example 20 is a method, comprising: administering at least two autoinjectors containing tranexamic acid, to a hemophiliac in preparation for a dental or medical procedure, and or following a dental or medical procedure.

Still further embodiments and examples are disclosed herein.

Example 21 is an at least one injectable formulation of tranexamic acid, or a pharmaceutically acceptable salt, ester, analog or derivative thereof, comprising at least 150 mg/mL of tranexamic acid.

Example 22, the subject matter of Example 21 additionally or alternatively includes, wherein the at least one injectable formulation of tranexamic acid further comprises about 167 mg/mL of tranexamic acid.

Example 23, the subject matter of Examples 21-22 additionally or alternatively includes, wherein the at least one injectable formulation of tranexamic acid further comprises more than 167 mg/mL of tranexamic acid. For example, the concentration can be 168 mg/mL or more than 168 mg/mL. In some embodiments, the concentration may approach 200 mg/mL or beyond 200 mg/mL. In one nonlimiting example, the concentration is about 222.2 mg/mL. These examples are not meant to be limiting.

Example 24, the subject matter of Examples 21-23 additionally or alternatively includes, wherein the at least one injectable formulation of tranexamic acid further comprises a supersaturated solution of tranexamic acid. In other examples, the formulation is a suspension of tranexamic acid. In still further embodiments, the formulation is a mixture or colloid containing tranexamic acid. In some nonlimiting examples, the concentration is about 222.2 mg/mL so that 0.5 grams of tranexamic acid are contained in about 2.25 mL of formulation, or so that 1 gram of tranexamic acid is contained in about 4.5 mL of formulation. A 0.5 gram tranexamic acid dosage can be one 2.25 mL injection, while a 1 gram tranexamic acid dosage can comprise two 2.25 mL injections or one 4.5 mL injection. A prefilled syringe, cartridge, or autoinjector may contain one, two, or more such injections. These examples are not meant to be limiting.

Example 25, the subject matter of Examples 21-24 additionally or alternatively includes, wherein the at least one injectable formulation of tranexamic acid further comprises at least one excipient ingredient in addition to water.

Example 26, the subject matter of Examples 21-25 additionally or alternatively includes, wherein the at least one injectable formulation of tranexamic acid further comprises at least one excipient ingredient selected from solvents, tonicity agents, chelators, buffers, pH adjusting agents, solubility enhancing agents, preservatives, or a combination thereof. Water is an aqueous solvent. Various other solvents exist and can be employed as pharmaceutically acceptable solvents including organic and inorganic solvents, glycerin, polyethylene glycol(s), other glycols, alcohols, e.g., ethanol, butanol, ketones, e.g., acetone, ethers, e.g., cyclopentyl methyl ether, oils, fatty acids and carboxylic acids, e.g., octanoic acid, solvent disulfides, and eutectic solvents. These examples are not meant to be limiting. Tonicity agents and salts can include sodium chloride and potassium chloride, and possibly sugars. These examples are not meant to be limiting. Chelators can include ethylenediaminetetraacetic acid (EDTA). These examples are not meant to be limiting. Buffers can include citric acid buffers, acetic acid buffers and sodium phosphate buffers. These examples are not meant to be limiting. Agents that adjust pH (pH adjusting agents) can include sodium hydroxide, acetic acid, hydrochloric acid, calcium carbonate, and gluconic acid. These examples are not meant to be limiting. Solubility enhancing agents can include polymers and liposomes. These examples are not meant to be limiting. Suspending agents can include one or more polymers, such as carbomers, microcrystalline cellulose, methylcellulose, hydroxypropyl-methylcellulose, carboxymethylcellulose and its salts thereof. These examples are not meant to be limiting. Preservatives can include sodium benzoate, benzyl alcohol, metabisulfites, and chlorobutanol. These examples are not meant to be limiting. Various combinations of two or more of any of these excipients is possible and can be employed. Some embodiments of an at least one injectable formulation of tranexamic acid utilize a form of calcium and or a form of carbonate or bicarbonate as additional active pharmaceutical ingredient(s), such as calcium chloride, calcium gluconate, calcium carbonate, calcium bicarbonate, sodium carbonate, sodium bicarbonate, or any analog, derivative, combination or pharmaceutically acceptable salt thereof. These examples are not meant to be limiting. In some embodiments, the formulation is extended release, immediate release, or a combination thereof. One or more excipients or types of excipients can be used in combination in some various embodiments.

Example 27, the subject matter of Examples 21-26 additionally or alternatively includes, wherein the at least one injectable formulation of tranexamic acid further comprises at least one procoagulant agent or hemostatic agent in addition to tranexamic acid.

Example 28, the subject matter of Examples 21-27 additionally or alternatively includes, wherein the at least one injectable formulation of tranexamic acid further comprises at least one active pharmaceutical ingredient in addition to tranexamic acid.

Example 29, the subject matter of Examples 21-28 additionally or alternatively includes, wherein the at least one injectable formulation of tranexamic acid further comprises at least one sterile container.

Example 30, the subject matter of Examples 21-29 additionally or alternatively includes, wherein the at least one injectable formulation of tranexamic acid further comprises at least one sterile container selected from prefilled syringes, cartridges, autoinjectors, vials, ampoules, intravenous fluid bags, and bottles.

Example 31, the subject matter of Examples 21-30 additionally or alternatively includes, wherein the at least one injectable formulation of tranexamic acid further comprises a volume of at least 0.5 mL.

Example 32, the subject matter of Examples 21-31 additionally or alternatively includes, wherein the at least one injectable formulation of tranexamic acid further comprises a volume of at least 1 mL.

Example 33, the subject matter of Examples 21-32 additionally or alternatively includes, wherein the at least one injectable formulation of tranexamic acid further comprises a volume of at least 3 mL.

Example 34, the subject matter of Examples 21-33 additionally or alternatively includes, wherein the at least one injectable formulation of tranexamic acid further comprises a volume of at least 5 mL.

Example 35, the subject matter of Examples 21-34 additionally or alternatively includes, wherein the at least one injectable formulation of tranexamic acid further comprises a volume of at least 6 mL.

Example 36, the subject matter of Examples 21-35 additionally or alternatively includes, wherein the at least one injectable formulation of tranexamic acid further comprises a volume of at least 10 mL.

Example 37, the subject matter of Examples 21-36 additionally or alternatively includes, wherein the at least one injectable formulation of tranexamic acid further comprises a volume of at least 20 mL.

Example 38, the subject matter of Examples 21-37 additionally or alternatively includes, wherein the at least one injectable formulation of tranexamic acid further comprises a volume of more than 21 mL.

Example 39, the subject matter of Examples 21-38 additionally or alternatively includes, wherein the at least one injectable formulation of tranexamic acid further comprises at least 0.5 gram of tranexamic acid or a pharmaceutically acceptable salt, ester, analog or derivative thereof.

Example 40, the subject matter of Examples 21-39 additionally or alternatively includes, wherein the at least one injectable formulation of tranexamic acid further comprises at least 1 gram of tranexamic acid or a pharmaceutically acceptable salt, ester, analog or derivative thereof.

Example 41, the subject matter of Examples 21-40 additionally or alternatively includes, wherein the at least one injectable formulation of tranexamic acid further comprises at least 3 grams of tranexamic acid or a pharmaceutically acceptable salt, ester, analog or derivative thereof.

Example 42, the subject matter of Examples 21-41 additionally or alternatively includes, wherein the at least one injectable formulation of tranexamic acid further comprises at least 5 grams of tranexamic acid or a pharmaceutically acceptable salt, ester, analog or derivative thereof.

Example 43, the subject matter of Examples 21-42 additionally or alternatively includes, wherein the at least one injectable formulation of tranexamic acid further comprises at least 10 grams of tranexamic acid or a pharmaceutically acceptable salt, ester, analog or derivative thereof.

Example 44, the subject matter of Examples 21-43 additionally or alternatively includes, wherein the at least one injectable formulation of tranexamic acid further comprises at least 20 grams of tranexamic acid or a pharmaceutically acceptable salt, ester, analog or derivative thereof.

Example 45, the subject matter of Examples 21-44 additionally or alternatively includes, wherein the at least one injectable formulation of tranexamic acid further comprises more than 21 grams of tranexamic acid or a pharmaceutically acceptable salt, ester, analog or derivative thereof.

Example 46, the subject matter of Examples 21-45 additionally or alternatively includes, wherein the at least one injectable formulation of tranexamic acid further comprises no more than 10% impurities at release or over its shelf life.

Example 47, the subject matter of Examples 21-46 additionally or alternatively includes, wherein the at least one injectable formulation of tranexamic acid further comprises a shelf life of at least 12 months.

Example 48, the subject matter of Examples 21-47 additionally or alternatively includes, wherein the at least one injectable formulation of tranexamic acid further comprises a dry or concentrated form of tranexamic acid requiring reconstitution or dilution with a solvent prior to use. In some examples and embodiments, an anhydrous or lyophilized form of tranexamic acid or a formulation thereof is employed. In some embodiments and examples, a dual chamber container contains a concentrated or dry powder form or formulation of tranexamic acid in one chamber and a solvent for diluting or reconstituting said concentrated or dry powder form or formulation of tranexamic acid, before mixing. Alternatively, one container holds the concentrated or dry powder form or formulation of tranexamic acid.

Example 49, the subject matter of Examples 21-48 additionally or alternatively includes, a method comprising administering at least one injection of said at least one injectable formulation of tranexamic acid, or a pharmaceutically acceptable salt, ester, analog or derivative thereof, to a patient to reduce or prevent internal bleeding, external bleeding, or a combination thereof.

Example 50, the subject matter of Examples 21-49 additionally or alternatively includes, wherein at least two injections of said at least one injectable formulation of tranexamic acid are administered to a patient.

Example 51, the subject matter of Examples 21-50 additionally or alternatively includes, wherein at least one injection of said at least one injectable formulation of tranexamic acid is administered to a patient by intramuscular injection.

Example 52, the subject matter of Examples 21-51 additionally or alternatively includes, wherein at least two injections of said at least one injectable formulation of tranexamic acid are administered to a patient by intramuscular injection.

Example 53, the subject matter of Examples 21-52 additionally or alternatively includes, wherein at least one injection of said at least one injectable formulation of tranexamic acid is administered to a patient by intramuscular injection, wherein at least another injection of said at least one injectable formulation of tranexamic acid is administered intravenously by bolus or infusion.

Example 54, the subject matter of Examples 21-53 additionally or alternatively includes, wherein at least one injection of said at least one injectable formulation of tranexamic acid is administered intravenously by bolus or infusion.

Example 55, the subject matter of Examples 21-54 additionally or alternatively includes, wherein said patient is a patient that is suspected of being injured and bleeding, is injured and bleeding, or is at high risk of bleeding.

Example 56, the subject matter of Examples 21-55 additionally or alternatively includes, wherein said patient is a postpartum female patient to treat or prevent postpartum hemorrhage.

Example 57, the subject matter of Examples 21-56 additionally or alternatively includes, wherein said patient is a traumatic injury patient.

Example 58, the subject matter of Examples 21-57 additionally or alternatively includes, wherein said patient is a hemophiliac or a patient on blood thinner medication.

Example 59, the subject matter of Examples 21-58 additionally or alternatively includes, wherein said at least one injection of said at least one injectable formulation of tranexamic acid is administered to said patient prior to a surgical procedure, during a surgical produce, after a surgical procedure, or a combination thereof.

Example 60, the subject matter of Examples 21-59 additionally or alternatively includes, wherein said method further comprises the step of reconstituting or diluting said at least one injection of said at least one injectable formulation of tranexamic acid with a solvent prior to use.

Example 61, the subject matter of Examples 21-60 additionally or alternatively includes, an autoinjector containing up to two prefilled syringes or cartridges each containing 5 to 10 mL of an at least one injectable formulation of tranexamic acid, or a pharmaceutically acceptable salt, ester, analog or derivative thereof, at a concentration of least 150 mg/mL of tranexamic acid. In other embodiments and examples, said autoinjector contains up to two prefilled syringes or cartridges each contain 2 to 5 mL of an at least one injectable formulation of tranexamic acid, or a pharmaceutically acceptable salt, ester, analog or derivative thereof, at a concentration of least 150 mg/mL of tranexamic acid.

Example 62, the subject matter of Examples 21-61 additionally or alternatively includes, wherein said autoinjector is waterproof and or impact resistant for military use and transport.

Example 63, the subject matter of Examples 21-62 additionally or alternatively includes, wherein said autoinjector is contained in a waterproof and or impact resistant case for military use and transport.

Example 64, the subject matter of Examples 21-63 additionally or alternatively includes, a prefilled syringe or injectable cartridge containing 5 to 10 mL of an at least one injectable formulation of tranexamic acid, or a pharmaceutically acceptable salt, ester, analog or derivative thereof, at a concentration of least 150 mg/mL of tranexamic acid and transport.

Example 65, the subject matter of Examples 21-64 additionally or alternatively includes, wherein said prefilled syringe or injectable cartridge is further contained in a waterproof and or impact resistant case for military use and transport.

Still further embodiments and examples are disclosed herein.

Example 66 is an at least one injectable formulation of tranexamic acid, or a pharmaceutically acceptable salt, ester, analog or derivative thereof, comprising at least 100 mg/mL of tranexamic acid inside an at least one prefilled syringe container or cartridge thereof. In many embodiments, this is a liquid injectable formulation of tranexamic acid.

Example 67, the subject matter of Example 66 additionally or alternatively includes, wherein further comprising at least a half gram of tranexamic acid inside an at least 5 mL filled prefilled syringe or cartridge thereof, preferably in a 5 mL prefilled syringe.

Example 68, the subject matter of Examples 66-67 additionally or alternatively includes, wherein further comprising at least one gram of tranexamic acid inside an at least 10 mL filled prefilled syringe or cartridge thereof, preferably in a 10 mL prefilled syringe.

Example 69, the subject matter of Examples 66-68 additionally or alternatively includes, wherein further comprising at least about a half gram of tranexamic acid in a 3 mL filled 3 mL prefilled syringe or cartridge thereof.

Example 70, the subject matter of Examples 66-69 additionally or alternatively includes, wherein further comprising at least about a half gram of tranexamic acid in a 3 mL filled 5 mL prefilled syringe or cartridge thereof.

Example 71, the subject matter of Examples 66-70 additionally or alternatively includes, wherein further comprising at least about 0.83 grams of tranexamic acid in a 5 mL filled 5 mL prefilled syringe or cartridge thereof.

Example 72, the subject matter of Examples 66-71 additionally or alternatively includes, wherein further comprising at least about a gram of tranexamic acid in a 6 mL filled 6 mL prefilled syringe or cartridge thereof.

Example 73, the subject matter of Examples 66-72 additionally or alternatively includes, wherein further comprising at least about a gram of tranexamic acid in a 6 mL filled 10 mL prefilled syringe or cartridge thereof.

Example 74, the subject matter of Examples 66-73 additionally or alternatively includes, wherein further comprising at least about 1.67 grams of tranexamic acid in a 10 mL filled 10 mL prefilled syringe or cartridge thereof.

Example 75, the subject matter of Examples 66-74 additionally or alternatively includes, wherein further comprising a supersaturated solution, suspension, or colloid of tranexamic acid.

Example 76, the subject matter of Examples 66-75 additionally or alternatively includes, wherein further comprising at least one excipient ingredient in addition to water.

Example 77, the subject matter of Examples 66-76 additionally or alternatively includes, wherein further comprising at least one excipient ingredient selected from solvents, tonicity agents, chelators, buffers, pH adjusting agents, salts, solubility enhancing agents, suspending agents, dispersants, surfactants, crystallization inhibitors, freeze inhibitors, viscosity adjusting agents, stabilizers, preservatives, or a combination thereof.

Example 78, the subject matter of Examples 66-77 additionally or alternatively includes, wherein further comprising at least one procoagulant agent or hemostatic agent in addition to tranexamic acid.

Example 79, the subject matter of Examples 66-78 additionally or alternatively includes, wherein further comprising at least one active pharmaceutical ingredient in addition to tranexamic acid.

Example 80, the subject matter of Examples 66-79 additionally or alternatively includes, wherein further comprising at least one sterile container, prefilled syringe or cartridge thereof.

Example 81, the subject matter of Examples 66-80 additionally or alternatively includes, wherein further comprising a volume of at least 0.5 mL.

Example 82, the subject matter of Examples 66-81 additionally or alternatively includes, wherein further comprising a volume of at least 1 mL.

Example 83, the subject matter of Examples 66-82 additionally or alternatively includes, wherein further comprising a volume of at least 3 mL.

Example 84, the subject matter of Examples 66-83 additionally or alternatively includes, wherein further comprising a volume of at least 5 mL.

Example 85, the subject matter of Examples 66-84 additionally or alternatively includes, wherein further comprising a volume of at least 6 mL.

Example 86, the subject matter of Examples 66-85 additionally or alternatively includes, wherein further comprising a volume of at least 10 mL.

Example 87, the subject matter of Examples 66-86 additionally or alternatively includes, wherein further comprising a volume of at least 10.5 mL.

Example 88, the subject matter of Examples 66-87 additionally or alternatively includes, wherein further comprising a volume of at least 11 mL.

Example 89, the subject matter of Examples 66-88 additionally or alternatively includes, wherein further comprising no more than 10% impurities at release or over its shelf life.

Example 90, the subject matter of Examples 66-89 additionally or alternatively includes, wherein further comprising a shelf life of at least 12 months.

Example 91, the subject matter of Examples 66-90 additionally or alternatively includes, wherein further comprising a dry or concentrated form of tranexamic acid requiring reconstitution or dilution with a solvent prior to use.

Example 92, the subject matter of Examples 66-91 additionally or alternatively includes, a method comprising administering at least one injection from an at least one prefilled syringe of said at least one injectable formulation of tranexamic acid, or a pharmaceutically acceptable salt, ester, analog or derivative thereof, to a patient to reduce or prevent internal bleeding, external bleeding, or a combination thereof.

Example 93, the subject matter of Examples 66-92 additionally or alternatively includes, wherein further at least two injections of said at least one injectable formulation of tranexamic acid are administered to a patient from said at least one prefilled syringe.

Example 94, the subject matter of Examples 66-93 additionally or alternatively includes, wherein further at least one injection of said at least one injectable formulation of tranexamic acid is administered to a patient by intramuscular injection.

Example 95, the subject matter of Examples 66-94 additionally or alternatively includes, wherein further at least two injections of said at least one injectable formulation of tranexamic acid are administered to a patient by intramuscular injection.

Example 96, the subject matter of Examples 66-95 additionally or alternatively includes, wherein further at least one injection of said at least one injectable formulation of tranexamic acid is administered to a patient by intramuscular injection, wherein at least another injection of said at least one injectable formulation of tranexamic acid is administered intravenously by bolus or infusion.

Example 97, the subject matter of Examples 66-96 additionally or alternatively includes, wherein further at least one injection of said at least one injectable formulation of tranexamic acid is administered intravenously by bolus or infusion.

Example 98, the subject matter of Examples 66-97 additionally or alternatively includes, wherein further said patient is a patient that is suspected of being injured and bleeding, is injured and bleeding, or is at high risk of bleeding.

Example 99, the subject matter of Examples 66-98 additionally or alternatively includes, wherein further said patient is a postpartum female patient to treat or prevent postpartum hemorrhage or gastrointestinal bleeding.

Example 100, the subject matter of Examples 66-99 additionally or alternatively includes, wherein further said patient is a traumatic injury patient and or traumatic brain injury patient. In other embodiments, said patient has intracerebral hemorrhage or intracranial hemorrhage; either traumatic intracranial hemorrhage or spontaneous intracranial hemorrhage without traumatic injury.

Example 101, the subject matter of Examples 66-100 additionally or alternatively includes, wherein further said patient is a hemophiliac or a patient on blood thinner medication.

Example 102, the subject matter of Examples 66-101 additionally or alternatively includes, wherein further said at least one injection of said at least one injectable formulation of tranexamic acid is administered to said patient prior to a surgical procedure, during a surgical produce, after a surgical procedure, or a combination thereof.

Example 103, the subject matter of Examples 66-102 additionally or alternatively includes, wherein further comprising the step of reconstituting or diluting said at least one injection of said at least one injectable formulation of tranexamic acid with a solvent prior to use.

Example 104, the subject matter of Examples 66-103 additionally or alternatively includes, a prefilled syringe or injectable cartridge containing 3 to 10 mL of an at least one injectable formulation of tranexamic acid, or a pharmaceutically acceptable salt, ester, analog or derivative thereof, at a concentration of at least 100 mg/mL of tranexamic acid. This prefilled syringe may be injected by a user manually or with an injection assist device.

Example 105, the subject matter of Examples 66-104 additionally or alternatively includes, wherein further contained in a waterproof and or impact resistant case for use and transport or military use and transport.

Example 106, the subject matter of Examples 66-105 additionally or alternatively includes, wherein further comprising at least one excipient ingredient in addition to water.

Example 107, the subject matter of Examples 66-106 additionally or alternatively includes, wherein further comprising at least one excipient ingredient selected from solvents, tonicity agents, chelators, buffers, pH adjusting agents, salts, solubility enhancing agents, suspending agents, dispersants, surfactants, crystallization inhibitors, freeze inhibitors, viscosity adjusting agents, stabilizers, preservatives, or a combination thereof.

Example 108, the subject matter of Examples 66-107 additionally or alternatively includes, wherein further containing at least two dosages of tranexamic acid.

The above examples are not meant to be limiting and other embodiments are permitted within the scope of this invention.

One primary embodiment of the invention is a prefilled syringe or injectable cartridge containing 1 mL to 10 mL of an at least one injectable formulation of tranexamic acid, or a pharmaceutically acceptable salt, ester, analog or derivative thereof, at a concentration of at least 100 mg/mL of tranexamic acid. The at least one injectable formulation of tranexamic acid further comprises about 125 mg/mL of tranexamic acid or alternatively may comprise about 150 mg/mL of tranexamic acid, about 167 mg/mL of tranexamic acid, or more than 167 mg/mL of tranexamic acid. In some embodiments, the at least one injectable formulation of tranexamic acid comprises a supersaturated solution, suspension, or colloid of tranexamic acid. In such embodiments having a supersaturated solution, suspension, or colloid of tranexamic acid, the concentration may be well beyond 167 mg/mL, such as greater than 170 mg/mL, or even greater than 200 mg/mL. In some nonlimiting examples, the concentration is about 222.2 mg/mL so that 0.5 grams of tranexamic acid are contained in about 2.25 mL of formulation, or so that 1 gram of tranexamic acid is contained in about 4.5 mL of formulation. A 0.5 gram tranexamic acid dosage can be one 2.25 mL injection, while a 1 gram tranexamic acid dosage can comprise two 2.25 mL injections or one 4.5 mL injection. In another nonlimiting example, a 0.5 gram tranexamic acid dosage can be one 3 mL injection, while a 1 gram tranexamic acid dosage can comprise two 3 mL injections or one 6 mL injection. These examples are not meant to be limiting. Other nonlimiting examples of concentrations of tranexamic acid, or a pharmaceutically acceptable salt, ester, analog or derivative thereof, can include one gram in 5 mL, one gram in 4 mL, one gram in 3 mL, one gram in 2.5 mL, one gram in 2 mL, and even one gram in 1 mL; or alternatively, 0.5 grams in 2.5 mL, 0.5 grams in 2 mL, 0.5 grams in 1.5 mL, 0.5 grams in 1.25 mL, 0.5 grams in 1 mL, and even 0.5 grams in 0.5 mL. The at least one injectable formulation of tranexamic acid, or a pharmaceutically acceptable salt, ester, analog or derivative thereof, in some embodiments has a viscosity below 30 centipoise; in other embodiments, has a viscosity between 30 and 60 centipoise; and in still other embodiments, has a viscosity above 60 centipoise.

In many embodiments and methods for intramuscular injection, the gauge of the needle selected, either Luer-fitting or pre-staked or otherwise, accommodates the strength, speed, and gauge needed for efficient intramuscular injection, including in some embodiments, intramuscular injection of higher viscosity formulations. In most embodiments, the needle is able to pierce clothing.

In other embodiments and methods, the prefilled syringe or injectable cartridge has a Luer-fitting adapted for intravenous delivery, either with an intravenous catheter, intravenous line, or infusion bag.

Many embodiments comprise an aqueous solution of tranexamic acid.

Some embodiments further comprise an at least one excipient ingredient in addition to sterile water. Some embodiments further contain an at least one excipient ingredient selected from solvents, tonicity agents, chelators, buffers, pH adjusting agents, salts, solubility enhancing agents, suspending agents, dispersants, surfactants, crystallization inhibitors, freeze inhibitors, viscosity adjusting agents, stabilizers, preservatives, or a combination thereof. These types of excipient examples are not meant to be limiting.

Other embodiments further comprise an at least one procoagulant agent or hemostatic agent in addition to tranexamic acid.

Other embodiments further comprise an at least one active pharmaceutical ingredient in addition to tranexamic acid. For example, some embodiments further comprise an at least one active pharmaceutical ingredient in addition to tranexamic acid selected from a form of calcium, calcium chloride, calcium gluconate, calcium carbonate, calcium bicarbonate, a form of carbonate, calcium carbonate, sodium carbonate, a form of bicarbonate, calcium bicarbonate, sodium bicarbonate, or any analog, derivative, combination or pharmaceutically acceptable salt thereof. These examples are not meant to be limiting.

Some embodiments comprise at least about a half gram of tranexamic acid in a 1 mL to 3 mL of fill volume of said prefilled syringe or injectable cartridge. For example, this can include a 2.25 mL of fill volume or a 3 mL of fill volume. Other fill volumes are possible. Other embodiments comprise at least about a half gram of tranexamic acid in 5 mL of fill volume of said prefilled syringe or injectable cartridge. Still other embodiments comprise at least about one gram of tranexamic acid in a 4 mL to 6 mL of fill volume of said prefilled syringe or injectable cartridge thereof. For example, this can include a 4.5 mL of fill volume or a 5 mL fill volume. Other fill volumes are possible. Still even further embodiments comprise at least about one gram of tranexamic acid in 10 mL of fill volume of said prefilled syringe or injectable cartridge thereof. These embodiment examples are not meant to be limiting and other embodiments of different tranexamic acid quantities contained in different volumes of fill exist.

In many embodiments, the at least one injectable formulation of tranexamic acid comprises no more than 10% impurities at release or over its shelf life.

In many embodiments, the at least one injectable formulation of tranexamic acid comprises a shelf life of at least 12 months.

In some embodiments, the at least one injectable formulation of tranexamic acid comprises a dry or concentrated form of tranexamic acid requiring reconstitution or dilution with a solvent prior to use. The tranexamic acid can be contained in a separate container or vial from the solvent and is then combined or mixed together prior to use. In some embodiments, a dual chamber or multi-chamber container, syringe, cartridge, or vial is used.

In some embodiments, the prefilled syringe or injectable cartridge contains at least two dosages of tranexamic acid.

In some embodiments, the prefilled syringe or injectable cartridge is further contained in a waterproof and or impact resistant case for civilian use and transport, military use and transport, or a combination thereof. The case may contain one or more prefilled syringes or injectable cartridges, and may preferably include two to four such prefilled syringes or injectable cartridges.

In some embodiments, the prefilled syringe or injectable cartridge is further administered with the aid of a manual syringe assist device that said prefilled syringe or injectable cartridge fits into.

In some embodiments, the prefilled syringe or injectable cartridge is further administered with the aid of an autoinjector that contains said prefilled syringe or injectable cartridge.

In some embodiments, the prefilled syringe or injectable cartridge is further supplied in a kit containing at least two said prefilled syringes or injectable cartridges.

Another primary embodiment is an autoinjector providing an at least one half gram dosage of tranexamic acid in a 1 mL to 5 mL injection volume of an injectable formulation of tranexamic acid. For example, this can include a 2.25 mL injection volume or a 3 mL injection volume. In another primary embodiment, an autoinjector provides an at least one gram dosage of tranexamic acid in a 4 mL to 6 mL injection volume of an injectable formulation of tranexamic acid. For example, this can include a 4.5 mL injection volume or a 6 mL injection volume. Other injection volumes are possible and these examples are not meant to be limiting. The at least one injectable formulation of tranexamic acid, or a pharmaceutically acceptable salt, ester, analog or derivative thereof, in some embodiments has a viscosity below 30 centipoise; in other embodiments, has a viscosity between 30 and 60 centipoise; and in still other embodiments, has a viscosity above 60 centipoise. In many embodiments, the gauge of the needle utilized accommodates the strength, speed, and gauge needed for efficient intramuscular injection, including in some embodiments, intramuscular injection of higher viscosity formulations. In most embodiments, the needle is able to pierce clothing.

In some embodiments, the autoinjector further comprises a dry or concentrated form of tranexamic acid requiring reconstitution or dilution with a solvent prior to use. The tranexamic acid can be contained in a separate container or vial from the solvent and is then combined or mixed together prior to use. In some embodiments, a dual chamber or multi-chamber container, syringe, cartridge, or vial is used.

In some embodiments, the autoinjector is or is made to be impact resistant, waterproof, or a combination thereof, and optionally includes a case. The case can further provide impact resistance, waterproofing, or a combination thereof. The case may contain one or more autoinjectors, and may preferably include two to four such autoinjectors.

In many embodiments, the autoinjector provides intramuscular injection of at least one half gram dosage of tranexamic acid. Some embodiments of autoinjector provide subcutaneous injection of at least one half gram dosage of tranexamic acid.

Another primary embodiment is a method comprising administering to a patient at least two at least half gram intramuscular dosages of tranexamic acid with an at least one prefilled syringe or injectable cartridge, wherein each of said at least two at least half gram intramuscular dosages of tranexamic acid have an injection volume of 1 mL to 5 mL of an injectable formulation of tranexamic acid, to reduce or prevent internal bleeding, external bleeding, reduce blood transfusion(s), or a combination thereof. For example, this can include a 2.25 mL injection volume or a 3 mL injection volume. Other injection volumes are possible and these examples are not meant to be limiting. Some embodiments provide subcutaneous injection of tranexamic acid. Some embodiments provide at least two at least half gram subcutaneous dosages of tranexamic acid have an injection volume of 1 mL to 5 mL of an injectable formulation of tranexamic acid. For example, this can include a 2.25 mL injection volume or a 3 mL injection volume. Other injection volumes are possible and these examples are not meant to be limiting. This method further may optionally or alternatively administer said at least two at least half gram intramuscular dosages of tranexamic acid as an at least one at least one gram intramuscular dosage of tranexamic acid with an at least one prefilled syringe or injectable cartridge. For example, this at least one at least one gram intramuscular dosage of tranexamic acid can include a 4 mL to 6 mL injection volume, such as a 4.5 mL injection volume. This example is not meant to be limiting.

In many embodiments of this method, the patient has or is suspected of having at least one condition selected from bleeding, gastrointestinal bleeding, hemorrhagic wound, injury, traumatic injury, traumatic brain injury, intracerebral hemorrhage, intracranial hemorrhage, traumatic intracranial hemorrhage, spontaneous intracranial hemorrhage (without traumatic injury), high risk of bleeding, hemophilia, surgery, post-surgery, will undergo surgery, has recently given birth, or a combination thereof.

In many embodiments, at least 0.5 grams of tranexamic acid, and preferably at least 1 gram of tranexamic acid, is administered by one or more prefilled syringes or injectable cartridges as soon as is possible after an injury is sustained. For example, immediately administered after an injury is sustained, or within 5 minutes, or within 10 minutes, or within 20 minutes, or within a half hour after an injury is sustained. In other embodiments, if not possible to administer the prefilled syringe(s) or injectable cartridge(s) right away, tranexamic acid should at least be administered within several hours after an injury is sustained, and preferably within one hour. In other embodiments, such as for soldiers preparing for battle, at least 0.5 grams of tranexamic acid, and preferably at least 1 gram of tranexamic acid, is prophylactically administered before sustaining a possible injury.

Another primary embodiment is a method comprising administering to a patient at least two at least half gram intramuscular dosages of tranexamic acid with an at least two autoinjectors, wherein each of said at least two at least half gram intramuscular dosages of tranexamic acid have an injection volume of 1 mL to 5 mL of an injectable formulation of tranexamic acid, to reduce or prevent internal bleeding, external bleeding, reduce blood transfusion(s), or a combination thereof. For example, this can include a 2.25 mL injection volume or a 3 mL injection volume. Other injection volumes are possible and these examples are not meant to be limiting. Some embodiments provide subcutaneous injection of tranexamic acid. Some embodiments provide at least two at least half gram subcutaneous dosages of tranexamic acid have an injection volume of 1 mL to 5 mL of an injectable formulation of tranexamic acid. For example, this can include a 2.25 mL injection volume or a 3 mL injection volume. Other injection volumes are possible and these examples are not meant to be limiting. This method further may optionally or alternatively administer said at least two at least half gram intramuscular dosages of tranexamic acid as an at least one at least one gram intramuscular dosage of tranexamic acid with an at least one autoinjector. For example, this at least one at least one gram intramuscular dosage of tranexamic acid can include a 4 mL to 6 mL injection volume, such as a 4.5 mL injection volume. This example is not meant to be limiting.

In many embodiments of this method, the patient has or is suspected of having at least one condition selected from bleeding, gastrointestinal bleeding, hemorrhagic wound, injury, traumatic injury, traumatic brain injury, intracerebral hemorrhage, intracranial hemorrhage, traumatic intracranial hemorrhage, spontaneous intracranial hemorrhage (without traumatic injury), high risk of bleeding, hemophilia, surgery, post-surgery, will undergo surgery, has recently given birth, or a combination thereof.

In many embodiments, at least 0.5 grams of tranexamic acid, and preferably at least 1 gram of tranexamic acid, is administered by one or more autoinjectors as soon as is possible after an injury is sustained. For example, immediately administered after an injury is sustained, or within 5 minutes, or within 10 minutes, or within 20 minutes, or within a half hour after an injury is sustained. In other embodiments, if not possible to administer the autoinjector or autoinjectors right away, tranexamic acid should at least be administered within several hours after an injury is sustained, and preferably within one hour. In other embodiments, such as for soldiers preparing for battle, at least 0.5 grams of tranexamic acid, and preferably at least 1 gram of tranexamic acid, is prophylactically administered before sustaining a possible injury.

In many of the methods embodiments described above, intramuscular injection site or sites are relevant. The location of the injury or wound or wounds that the soldier or patient is experiencing also comes into play in relation to those intramuscular injection site(s). In some method embodiments, an at least one intramuscular injection of an at least one injectable formulation of tranexamic acid, or a pharmaceutically acceptable salt, ester, analog or derivative thereof, is injected into the thigh(s), upper arm(s), buttocks, one or more other body areas, or a combination thereof. Muscle locations for intramuscular injection include the vastus lateralis muscle of the thigh, the deltoid muscle of the upper arm, and the gluteal muscle of the buttocks. These muscles and muscle locations are not meant to be limiting and other muscles can be used when necessary. In some method embodiments, two or more intramuscular injections take place on the left side of the body only. In some method embodiments, two or more intramuscular injections take place on the right side of the body only. In some method embodiments, an at least one intramuscular injection takes place on the left side of the body and an at least one intramuscular injection takes place on the right side of the body. In some method embodiments, two or more intramuscular injections take place on the lower body only. In some method embodiments, two or more intramuscular injections take place on the upper body only. In some method embodiments, an at least one intramuscular injection takes place on the lower body and an at least one intramuscular injection takes place on the upper body. For example, one intramuscular injection of an injectable formulation of tranexamic acid (e.g., one half gram or another amount of tranexamic acid) is administered into the left thigh and another is administered to the right thigh. In another example, one intramuscular injection of an injectable formulation of tranexamic acid (e.g., one half gram or another amount of tranexamic acid) is administered into the left upper arm and another is administered to the right upper arm. In another example, one intramuscular injection of an injectable formulation of tranexamic acid (e.g., one half gram or another amount of tranexamic acid) is administered into the left thigh and another is administered to the right upper arm. In another example, one intramuscular injection of an injectable formulation of tranexamic acid (e.g., one half gram or another amount of tranexamic acid) is administered into the right thigh and another is administered to the left upper arm. In another example, one intramuscular injection of an injectable formulation of tranexamic acid (e.g., one half gram or another amount of tranexamic acid) is administered into the left thigh, right thigh, left upper arm, and right upper arm. If legs or arms are severely wounded or missing, the gluteal muscles can be intramuscularly injected instead of a thigh and or upper arm, such as an intramuscular injection into the left and or right gluteal muscles. In another example, one intramuscular injection of an injectable formulation of tranexamic acid (e.g., one half gram or another amount of tranexamic acid) is administered into the left gluteal muscle, right gluteal muscle, left upper arm, and right upper arm. This example can apply for instance if a soldier is missing parts of the leg below the knee. In other examples, one or more injections are administered into the back of the soldier or patient or into the abdomen or chest of the soldier or patient, or a combination thereof. Non-intramuscular injections are also possible. These examples are not meant to be limiting. In some embodiments, these intramuscular injections occur nearly simultaneous. In other embodiments, these intramuscular injections occur sequentially with at least some time lapsed in between intramuscular injections. In some embodiments, one or more injections are repeated in the same injection site or a close vicinity of a previous injection site. In other embodiments, an injection is never repeated at or near a previous injection site or previously injected appendage. In some embodiments, these intramuscular injections occur only with prefilled syringes or injectable cartridges. In other embodiments, these intramuscular injections occur with only autoinjectors. In other embodiments, these intramuscular injections occur with a combination of prefilled syringes or injectable cartridges and autoinjectors. In still further embodiments, these intramuscular injection(s) are administered in combination with intravenous injection or infusion of tranexamic acid and or intraosseous injection. These embodiment examples are not meant to be limiting.

A still another primary embodiment is an injectable formulation of tranexamic acid, or a pharmaceutically acceptable salt, ester, analog or derivative thereof, comprising at least 125 mg/mL of tranexamic acid. Other embodiments comprise an injectable formulation of tranexamic acid of about 150 mg/mL of tranexamic acid, about 167 mg/mL of tranexamic acid, or more than 167 mg/mL of tranexamic acid. In some embodiments, the at least one injectable formulation of tranexamic acid comprises a supersaturated solution, suspension, or colloid of tranexamic acid. In such embodiments having a supersaturated solution, suspension, or colloid of tranexamic acid, the concentration may be well beyond 167 mg/mL, such as greater than 170 mg/mL, or even greater than 200 mg/mL. In one nonlimiting example, the concentration is about 222.2 mg/mL. In some nonlimiting examples, the concentration is about 222.2 mg/mL so that 0.5 grams of tranexamic acid are contained in about 2.25 mL of formulation, or so that 1 gram of tranexamic acid is contained in about 4.5 mL of formulation. A 0.5 gram tranexamic acid dosage can be one 2.25 mL injection, while a 1 gram tranexamic acid dosage can comprise two 2.25 mL injections or one 4.5 mL injection. In another nonlimiting example, a 0.5 gram tranexamic acid dosage can be one 3 mL injection, while a 1 gram tranexamic acid dosage can comprise two 3 mL injections or one 6 mL injection. These examples are not meant to be limiting. The injectable formulation of tranexamic acid can be contained in a vial, ampoule, syrette, prefilled syringe or injectable cartridge, or other pharmaceutically acceptable (sterile) container closure. The at least one injectable formulation of tranexamic acid, or a pharmaceutically acceptable salt, ester, analog or derivative thereof, in some embodiments has a viscosity below 30 centipoise; in other embodiments, has a viscosity between 30 and 60 centipoise; and in still other embodiments, has a viscosity above 60 centipoise.

The above embodiment examples are not meant to be limiting and other embodiments are permitted within the scope of this invention.

In many embodiments above, a functional analog and or functional derivative of tranexamic acid, and or even other functional polymorphs, can be used in place of tranexamic acid. In some other embodiments, this can include a hybrid molecule or hybrid compound containing a tranexamic acid moiety or tranexamic derivative moiety linked to or conjoined to one or more different molecules or compounds or moieties thereof. The one or more different molecules or compounds or moieties thereof may have biological activity as an enzyme or catalyst, signaling molecule, or a cellular targeting or delivery moiety. The hybrid molecule or hybrid compound is dual functional or multifunctional. These embodiments and examples are not meant to be limiting and other variations are possible.

Various illustrative components, blocks, configurations, modules, and steps have been described above generally in terms of their functionality. Persons having ordinary skill in the art may implement the described functionality in varying ways for each particular application, but such implementation decisions should not be interpreted as causing a departure from the scope of the present disclosure.

The previous description of the disclosed embodiments is provided to enable a person skilled in the art to make or use the disclosed embodiments. Various modifications to these embodiments will be readily apparent to those skilled in the art, and the principles defined herein may be applied to other embodiments without departing from the scope of the disclosure. Thus, the present disclosure is not intended to be limited to the embodiments shown herein but is to be accorded the widest scope possible consistent with the principles and novel features as previously described.

What is claimed is:

1. A prefilled syringe or injectable cartridge comprising 1 mL to 10 mL of an at least one injectable formulation of tranexamic acid, or a pharmaceutically acceptable salt, ester, analog or derivative thereof, at a concentration of at least 100 mg/mL of tranexamic acid.

2. The at least one injectable formulation of tranexamic acid of claim 1 further comprising about 150 mg/mL of tranexamic acid.

3. The at least one injectable formulation of tranexamic acid of claim 1 further comprising about 167 mg/mL of tranexamic acid.

4. The at least one injectable formulation of tranexamic acid of claim 1 further comprising more than 167 mg/mL of tranexamic acid.

5. The at least one injectable formulation of tranexamic acid of claim 1 further comprising a supersaturated solution, suspension, or colloid of tranexamic acid.

6. The at least one injectable formulation of tranexamic acid of claim 1 further comprising at least one excipient ingredient in addition to sterile water.

7. The at least one injectable formulation of tranexamic acid of claim 1 further comprising at least one excipient ingredient selected from solvents, tonicity agents, chelators, buffers, pH adjusting agents, salts, solubility enhancing agents, suspending agents, dispersants, surfactants, crystallization inhibitors, freeze inhibitors, viscosity adjusting agents, stabilizers, preservatives, or a combination thereof.

8. The at least one injectable formulation of tranexamic acid of claim 1 further comprising at least one procoagulant agent or hemostatic agent in addition to tranexamic acid.

9. The at least one injectable formulation of tranexamic acid of claim 1 further comprising at least one active pharmaceutical ingredient in addition to tranexamic acid.

10. The at least one injectable formulation of tranexamic acid of claim 1 further comprising at least one active pharmaceutical ingredient in addition to tranexamic acid selected from a form of calcium, calcium chloride, calcium gluconate, calcium carbonate, calcium bicarbonate, a form of carbonate, calcium carbonate, sodium carbonate, a form of bicarbonate, calcium bicarbonate, sodium bicarbonate, or any analog, derivative, combination or pharmaceutically acceptable salt thereof.

11. The at least one injectable formulation of tranexamic acid of claim 1 further comprising at least about a half gram of tranexamic acid in a 1 mL to 3 mL of fill volume of said prefilled syringe or injectable cartridge.

12. The at least one injectable formulation of tranexamic acid of claim 1 further comprising at least about a half gram of tranexamic acid in 5 mL of fill volume of said prefilled syringe or injectable cartridge.

13. The at least one injectable formulation of tranexamic acid of claim 1 further comprising at least about one gram of tranexamic acid in a 4 mL to 6 mL of fill volume of said prefilled syringe or injectable cartridge thereof.

14. The at least one injectable formulation of tranexamic acid of claim 1 further comprising at least about one gram of tranexamic acid in 10 mL of fill volume of said prefilled syringe or injectable cartridge thereof.

15. The at least one injectable formulation of tranexamic acid of claim 1 further comprising no more than 10% impurities at release or over its shelf life.

16. The at least one injectable formulation of tranexamic acid of claim 1 further comprising a shelf life of at least 12 months.

17. The at least one injectable formulation of tranexamic acid of claim 1 further comprising a dry or concentrated form of tranexamic acid requiring reconstitution or dilution with a solvent prior to use.

18. The prefilled syringe or injectable cartridge of claim 1 further comprising at least two dosages of tranexamic acid.

19. The prefilled syringe or injectable cartridge of claim 1 further contained in a waterproof and or impact resistant case for civilian use and transport, military use and transport, or a combination thereof.

20. The prefilled syringe or injectable cartridge of claim 1 further administered with the aid of a manual syringe assist device that said prefilled syringe or injectable cartridge fits into.

21. The prefilled syringe or injectable cartridge of claim 1 further administered with the aid of an autoinjector that includes said prefilled syringe or injectable cartridge.

22. The prefilled syringe or injectable cartridge of claim 1 further supplied in a kit comprising at least two said prefilled syringes or injectable cartridges.

23. An autoinjector providing an at least one half gram dosage of tranexamic acid in a 1 mL to 5 mL injection volume of an injectable formulation of tranexamic acid, or a pharmaceutically acceptable salt, ester, analog or derivative thereof.

24. The autoinjector of claim 23 further comprising a dry or concentrated form of tranexamic acid requiring reconstitution or dilution with a solvent prior to use.

25. The autoinjector of claim 23 further being impact resistant, waterproof, or a combination thereof, and optionally includes a case.

26. The autoinjector of claim 23 further providing intramuscular injection of said at least one half gram dosage of tranexamic acid.

27. A method, comprising:
administering to a patient at least two at least half gram intramuscular dosages of tranexamic acid with an at least one prefilled syringe or injectable cartridge, wherein each of said at least two at least half gram intramuscular dosages of tranexamic acid have an injection volume of 1 mL to 5 mL of an injectable formulation of tranexamic acid, to reduce or prevent internal bleeding, external bleeding, reduce blood transfusion(s), or a combination thereof; said method further optionally or alternatively administering said at least two at least half gram intramuscular dosages of tranexamic acid as an at least one at least one gram intramuscular dosage of tranexamic acid with an at least one prefilled syringe or injectable cartridge.

28. The method of claim 27, wherein said patient has or is suspected of having at least one condition selected from bleeding, gastrointestinal bleeding, hemorrhagic wound, injury, traumatic injury, traumatic brain injury, intracerebral hemorrhage, intracranial hemorrhage, traumatic intracranial hemorrhage, spontaneous intracranial hemorrhage without traumatic injury, high risk of bleeding, hemophilia, surgery, post-surgery, will undergo surgery, has recently given birth, or a combination thereof.

29. A method, comprising:
administering to a patient at least two at least half gram intramuscular dosages of tranexamic acid with an at least two autoinjectors, wherein each of said at least two at least half gram intramuscular dosages of tranexamic acid have an injection volume of 1 mL to 5 mL of an injectable formulation of tranexamic acid, to reduce or prevent internal bleeding, external bleeding, reduce blood transfusion(s), or a combination thereof; said method further optionally or alternatively administering said at least two at least half gram intramuscular dosages of tranexamic acid as an at least one at least one gram intramuscular dosage of tranexamic acid with an at least one autoinjector.

30. The method of claim 29, wherein said patient has or is suspected of having at least one condition selected from bleeding, gastrointestinal bleeding, hemorrhagic wound, injury, traumatic injury, traumatic brain injury, intracerebral hemorrhage, intracranial hemorrhage, traumatic intracranial hemorrhage, spontaneous intracranial hemorrhage without traumatic injury, high risk of bleeding, hemophilia, surgery, post-surgery, will undergo surgery, has recently given birth, or a combination thereof.

31. An injectable formulation of tranexamic acid, or a pharmaceutically acceptable salt, ester, analog or derivative thereof, comprising at least 125 mg/mL of tranexamic acid.
